(19) United States
(12) Patent Application Publication
(10) Pub. No.: US 2013/0210084 A1
(43) Pub. Date: Aug. 15, 2013

(54) USE OF MAGNESIUM HYDROXIDE FOR PH ADJUSTMENT AND IMPROVED SACCHARIFICATION OF BIOMASS

(71) Applicants: Michael N. Guerini, Gilroy, CA (US); Manojkumar Patel, Brampton (CA); Daniel A. Lane, Visalia, CA (US)

(72) Inventors: Michael N. Guerini, Gilroy, CA (US); Manojkumar Patel, Brampton (CA); Daniel A. Lane, Visalia, CA (US)

(73) Assignee: Edeniq, Inc., Visalia, CA (US)

(21) Appl. No.: 13/606,799

(22) Filed: Sep. 7, 2012

Related U.S. Application Data
(60) Provisional application No. 61/532,484, filed on Sep. 8, 2011.

(51) Int. Cl. C12P 19/14

(52) U.S. Cl. CPC C12P 19/14
USPC 435/99

(57) ABSTRACT

The present invention provides methods of processing biomass containing a cellulosic material that include contacting the cellulosic material with magnesium hydroxide to adjust the pH of the material before and/or after a high temperature and high pressure pretreatment step. The use of magnesium hydroxide provides a safer alternative to using ammonium hydroxide for pH adjustment, a more stable, buffered environment for improved operability in pH control, with similar or improved conversion of biomass to fermentable sugars, and similar or improved reduction of inhibitors during the subsequent hydrolysis or saccharification process.

### CROSS-REFERENCE TO RELATED PATENT APPLICATIONS

The present application claims benefit of priority to U.S. Provisional Patent Application No. 61/532,484, filed Sep. 8, 2011, which is incorporated herein by reference in its entirety.

### STATEMENT AS TO RIGHTS TO INVENTIONS MADE UNDER FEDERALLY SPONSORED RESEARCH AND DEVELOPMENT

This invention was made with government support under Grant Number “Federal Opportunity DE-FOA-0000096” awarded by the Department of Energy. The government has certain rights in the invention.

### BACKGROUND OF THE INVENTION

Ethanol produced from non-food biomass that contains cellulose is called cellulosic ethanol. Non-food biomass includes agricultural products such as corn stover, corn cobs and other inedible waste parts of food plants; grasses such as switchgrass; and forestry biomass, such as wood and waste wood products. Cellulose in plants is contained in lignocellulose, which is difficult to break down into fermentable sugars. Therefore, the lignocellulose must first be treated to make the cellulose accessible to hydrolysis, for example by saccharification enzymes. After this pretreatment step, the pH of the biomass comprising the cellulose is adjusted to a value suitable for enzymatic hydrolysis of the cellulose to fermentable sugars such as glucose. Typically, the pH of the biomass is adjusted using an alkali such as ammonium hydroxide (NH₄OH).

The use of NH₄OH to adjust the pH of cellulosic biomass materials during and after the pretreatment stages has health hazards and does not provide quality pH control throughout the saccharification process. Existing solutions are to continually adjust pH through the saccharification and fermentation process or use other caustic materials for pH adjustment at the end of the pre-treatment stage. The methods described herein address these problems.

### BRIEF SUMMARY OF THE INVENTION

The invention provides methods for processing biomass containing a cellulosic material to produce fermentable sugars. The disclosed methods are useful, for example, to produce ethanol from cellulosic biomass. In one aspect, the methods comprise using a mild base such as magnesium hydroxide to adjust the pH of the biomass to a range that is suitable for hydrolysis of the cellulosic material by saccharification enzymes. In some embodiments, the method comprises adjusting the pH of the biomass before treatment of the biomass with high temperature and pressure. Thus, in some embodiments, the method for processing biomass containing a cellulosic material comprises (a) contacting the biomass having a pH below about 6.5 with a sufficient amount of magnesium hydroxide (Mg(OH)₂) to increase the pH of the biomass above pH 7.0, (b) treating the biomass comprising Mg(OH)₂ at an elevated temperature and pressure, and (c) contacting the treated biomass with saccharification enzymes under conditions sufficient to hydrolyze at least a portion of the cellulose to fermentable sugars. In some embodiments, the pH of the biomass is increased from a starting range of about pH 5.5 to 6.2 in step (a). In some embodiments, the pH of the biomass is increased to a range of about pH 7.0 to 8.0.

In some embodiments, the method further comprises contacting the biomass that was treated at elevated temperature and pressure (HPHT treated biomass) with a sufficient amount of Mg(OH)₂ to adjust the pH to about 4.0-6.0. In some embodiments, the HPHT treated biomass is contacted with a sufficient amount of Mg(OH)₂ to adjust the pH to about 4.0-6.0 prior to contacting the HPHT-treated, pH adjusted biomass with saccharification enzymes. In some embodiments, the biomass comprising Mg(OH)₂ is treated at a temperature greater than about 160 degrees F. In some embodiments, the biomass comprising Mg(OH)₂ is treated at a pressure of greater than about 120 psi. In some embodiments, the biomass contains less than 1% by weight of a metal carbonate. In some embodiments, the pH of the saccharification step is maintained between about 4.7 and 5.0. In some embodiments, the biomass is contacted with yeast cells that express the saccharification enzymes.

In some embodiments, the particle size of the biomass is mechanically reduced either before, during or after the HPHT treatment step. In some embodiments, the biomass particle size is mechanically reduced with a colloidal mill. In some embodiments, the biomass particle size is reduced such that at least 95% of the biomass particles by weight have a particle size from about 100 microns to about 800 microns. In some embodiments, the biomass particle size is reduced such that at least 80%, at least 85%, at least 90%, or at least 95% of the biomass particles by weight have a particle size from about 100 microns to about 800 microns. In some embodiments, the biomass particle size is reduced such that at least 85% of the biomass particles by weight have a particle size from about 100 microns to about 800 microns. In some embodiments, the biomass particle size is reduced such that at least 80%, at least 85%, at least 90%, or at least 95% of the biomass particles by weight have a particle size from about 100 microns to about 500 microns. In some embodiments, the biomass particle size is reduced such that at least 85% of the biomass particles by weight have a particle size from about 100 microns to about 500 microns. In some embodiments, the biomass particle size is reduced such that at least 80%, at least 85%, at least 90%, or at least 95% of the biomass particles by weight have a particle size from about 50 microns to about 350 microns. In some embodiments, the biomass particle size is reduced such that at least 85% of the biomass particles by weight have a particle size from about 50 microns to about 350 microns.

In an another aspect, the invention provides a method for processing biomass containing a cellulosic material that does not require an acid pretreatment step. Thus, in some embodiments, an acid-free method is provided, the method comprising (a) contacting the biomass with magnesium hydroxide (Mg(OH)₂) at a concentration sufficient to raise the pH of the biomass at least 0.2 pH units to between pH 4.0 and 8.0, (b) treating the biomass comprising Mg(OH)₂ at an elevated temperature and pressure, and (c) contacting the treated biomass following steps (a) and (b) with saccharification enzymes under conditions sufficient to convert at least a portion of the cellulose to fermentable sugars. In some embodiments, step (a) occurs before step (b), and step (a) increases the pH of the biomass at least 0.5 pH units to between pH 7.0-8.0. In some embodiments, step (b) occurs before step (a), and step a) increases the pH of the biomass at least 0.5 pH units to between pH 3.5-5.0. In some embodiments, the method further comprises contacting the biomass with ammonium hydroxide (NH₄OH). In some embodiments, the biomass comprising Mg(OH)₂ is treated at a temperature greater than about 160 degrees F. In some embodiments, the biomass comprising Mg(OH)₂ is treated at a pressure of greater than about 120 psi. In some embodiments, the particle size of the biomass is mechanically reduced either before, during or after the HPHT treatment step. In some embodiments, the biomass particle size is mechanically reduced with a colloidal mill. In some embodiments, the biomass particle size is reduced such that at least 95% of the biomass particles by weight have a particle size from about 100 microns to about 800 microns. In some embodiments, the biomass particle size is reduced such that at least 80%, at least 85%, at least 90%, or at least 95% of the biomass particles by weight have a particle size from about 100 microns to about 800 microns. In some embodiments, the biomass particle size is reduced such that at least 85% of the biomass particles by weight have a particle size from about 100 microns to about 800 microns. In some embodiments, the biomass particle size is reduced such that at least 80%, at least 85%, at least 90%, or at least 95% of the biomass particles by weight have a particle size from about 100 microns to about 500 microns. In some embodiments, the biomass particle size is reduced such that at least 85% of the biomass particles by weight have a particle size from about 100 microns to about 500 microns. In some embodiments, the biomass particle size is reduced such that at least 80%, at least 85%, at least 90%, or at least 95% of the biomass particles by weight have a particle size from about 50 microns to about 350 microns. In some embodiments, the biomass particle size is reduced such that at least 85% of the biomass particles by weight have a particle size from about 50 microns to about 350 microns.

### DEFINITIONS

Unless defined otherwise, all technical and scientific terms used herein have the same meaning as commonly understood by one of ordinary skill in the art to which this invention pertains. Although essentially any methods and materials similar to those described herein can be used in the practice or testing of the present invention, only exemplary methods and materials are described. For purposes of the present invention, the following terms are defined below.

The terms “a,” “an,” and “the” include plural referents, unless the context clearly indicates otherwise.

The term “biomass” or “biomass feedstock” refers to any material comprising lignocellulosic material. Lignocellulosic materials are composed of three main components: cellulose, hemicellulose, and lignin. Cellulose and hemicellulose contain carbohydrates including polysaccharides and oligosaccharides, and can be combined with additional components, such as protein and/or lipid. Examples of biomass include agricultural products such as corn stover, corn cobs and other inedible waste parts of food plants; grasses such as switchgrass; and forestry biomass, such as wood and waste wood products.

The term “lignocellulosic” refers to material comprising both lignin and cellulose, and may also contain hemicellulose.

The term “cellulosic,” in reference to a material or composition, refers to a material comprising cellulose.

The term “saccharification” refers to production of fermentable sugars from polysaccharides by hydrolytic enzymes. Examples of hydrolytic enzymes include cellulase and hemicellulase. Hydrolytic enzymes are also referred to as “saccharification enzymes.”

The term “fermentable sugar” refers to a sugar that can be converted to ethanol during fermentation, for example during fermentation by yeast. For example, glucose is a fermentable sugar derived from hydrolysis of cellulose, whereas xylose, arabinose, mannose and galactose are fermentable sugars derived from hydrolysis of hemicellulose.

The term “simultaneous saccharification and fermentation (SSF) refers to providing saccharification enzymes during the fermentation process. This is in contrast to separate hydrolysis and fermentation (SHF) steps.

The term “pretreatment” refers to treating the biomass with physical, chemical or biological means, or any combination thereof, to render the biomass more susceptible to hydrolysis, for example, by saccharification enzymes. Pretreatment can consist of treating the biomass at high pressure and/or high temperature (HPHT). Pretreatment can further comprise physically mixing and/or milling the biomass in order to reduce the size of the biomass particles. Devices that are useful for physical pretreatment of biomass include, e.g., a hammermill, shear mill, cavitation mill or colloid or other high sheer mill. An exemplary colloid mill is the Cellunator™ (EdeniQ, Visalia, Calif.). Reduction of particle size is described in, for example, WO2010/025171, which is incorporated by reference herein.

The term “pretreated biomass” refers to biomass that has been subjected to pretreatment to render the biomass more susceptible to hydrolysis.

The term “high pressure,” in the context of a high pressure and high temperature (HPHT) pretreatment step, refers to a pressure above atmospheric pressure, for example at least 20, 30, 40, 50, 60, 70, 80, 90, 100, 110, 120, 130, 140, or 150 psi or greater.

The term “high temperature,” in the context of a high pressure and high temperature (HPHT) pretreatment step, refers to a temperature above ambient temperature, for example at least 100, 110, 120, 130, 140, 150, 160, 170, 180, 190, or 200 degrees F. or greater. When used in HPHT pretreatment, the term includes temperatures sufficient to substantially increase the pressure in a closed system. For example, the temperature in a closed system can be increased such that the pressure is at least 100 psi or greater, such as 110, 120, 130, 140, 150 psi or greater.

The term “hydrolysis” refers to breaking the glycosidic bonds in polysaccharides to yield simple monomeric and/or oligomeric sugars. For example, hydrolysis of cellulose produces the six carbon (C6) sugar glucose, whereas hydrolysis of hemicellulose produces the five carbon (C5) sugars xylose and arabinose. Hydrolysis can be accomplished by acid treatment or by enzymes such as cellulose, β-glucosidase, and xylanase.

The term “inhibitor” refers to a compound that inhibits the fermentation process. Inhibitors can be sugar degradation products that result from pretreatment of lignocellulose and/or cellulose. Examples of inhibitors include 2-furoic acid, 5-HMF, furfural, 4-HBA, syringic acid, vanillin, syringaldehyde, p-coumaric acid, and ferulic acid.

### BRIEF DESCRIPTION OF THE DRAWINGS

FIG. 1 shows conversion of citrus wood biomass to C6 sugars when the pH was adjusted with ammonium hydroxide or magnesium hydroxide after pretreatment, as described in Example 2.

FIG. 2 shows pH control throughout the experiment when using ammonium hydroxide or magnesium hydroxide to adjust the pH of citrus wood biomass after pretreatment, as described in Example 2.

FIG. 3 shows inhibitor production during saccharification of corn stover biomass that was pH adjusted with magnesium hydroxide before HPHT pretreatment, as described in Example 3.

### DETAILED DESCRIPTION OF THE INVENTION

The present invention provides a method for processing biomass containing a cellulosic material using magnesium hydroxide (Mg(OH)₂) to adjust the pH of the biomass both before and after pretreatment of the biomass. Traditional methods use ammonium hydroxide (NH₄OH) to adjust the pH. The present invention allows the replacement of NH₄OH with the less noxious, weaker base Mg(OH)₂. Surprisingly, the use of Mg(OH)₂ has the advantage of producing a more stable pH environment during the saccharification and fermentation process, and maintains the pH in the optimal activity range for cellulose hydrolysis enzymes. Further, the use of Mg(OH)₂ provides a source of magnesium during saccharification and fermentation, which improves (increases) the yields of fermentable sugars.

### pH Adjustment Before HPHT

In one aspect, the method described herein comprises adjusting the pH of the biomass containing a cellulosic material with Mg(OH)₂ before the high pressure and high temperature (HPHT) pretreatment step, thereby producing a pH-adjusted biomass. In some embodiments, the unadjusted pH of the biomass prior to pretreatment can be in the range of about 5.0 to about 6.5, or about 5.5 to about 6.2. In some embodiments, the method comprises contacting the biomass with Mg(OH)₂ at a concentration sufficient to adjust the pH to a value above neutral pH, where neutral pH is considered about pH 7.0. Thus, in some embodiments, the pH of the starting biomass material is increased using Mg(OH)₂ to the range of about 7.0 to about 9.0, about 7.0 to about 8.0, or about 7.0 to about 7.5, before the HPHT pretreatment step.

After the pH is adjusted using Mg(OH)₂, the pH adjusted biomass is pretreated to render the lignocellulose and cellulose more susceptible to hydrolysis. In some embodiments, pretreatment comprises subjecting the biomass to HPHT in order to render the lignocellulose and cellulose accessible to enzymatic hydrolysis. In some embodiments, the temperature and pressure are increased to amounts and for a time sufficient to render the cellulose susceptible to hydrolysis. In some embodiments, the temperature of the biomass is increased at this stage to at least about 170° F. to about 200° F., about 170° F. to about 190° F., or about 180° F. to about 190° F. In other embodiments, the temperature is increased in a closed system in order to increase the pressure. In one embodiment, the temperature is increased in the closed system until the pressure is increased to about 125 to 145 psi. Persons of skill in the art will understand that the amount of temperature increase necessary to sufficiently increase the pressure will depend on various factors, such as the size and shape of the closed system. In some embodiments, pretreatment comprises any other method known in the art that renders lignocellulose and cellulose more susceptible to hydrolysis, for example, acid treatment, alkali treatment, and steam treatment, or combinations thereof.

During the HPHT pretreatment stage, the pH of the biomass typically decreases, which can result in a pH below the range for optimal activity of saccharification enzymes. The optimal pH depends on the particular enzyme used, but is usually between about 4.0 and 6.0. As described in the examples, adjusting the pH of the biomass using Mg(OH)₂ before HPHT pretreatment results in a pH within the optimal range after the HPHT pretreatment stage. For example, in some embodiments, the pH of the biomass before HPHT pretreatment is adjusted to be within the range of about 7.0 to 7.5 by the addition of Mg(OH)₂, which results in a biomass having a pH in the range of about 4.2 to 5.2 after HPHT pretreatment. In contrast, in some embodiments, the unadjusted pH of the biomass, without the addition of Mg(OH)₂, is in the range of about 5.5 to 6.2 before HPHT pretreatment, and is in the range of about 3.8 to 4.5 after HPHT pretreatment. Thus, the addition of Mg(OH)₂ before HPHT pretreatment results in a biomass having a higher pH range after HPHT pretreatment than a biomass that was not pretreated with Mg(OH)₂.

In other embodiments, the pH-adjusted (i.e., adjusted with Mg(OH)₂) biomass can be subjected to mixing and milling during the HPHT process. For example, the pH-adjusted biomass can be processed by a mechanical device that creates uniformly smaller particles without creating fines. Fines are extra small particles that create problems in separating the unfermented solids from the ethanol and water, in particular when using corn as the biomass. Examples of mechanical devices useful in the present methods include, e.g., a hammermill, shear mill, cavitation mill or colloid or other high sheer mill. An exemplary colloid mill is the Cellunator™ (EdeniQ, Visalia, Calif.). Mechanical reduction of particle size is described in, for example, WO2010/025171, which is incorporated by reference herein.

In some embodiments, the use of Mg(OH)₂ to adjust the pH of the biomass before pretreatment increases the conversion of cellulose to C6 sugars such as glucose during the subsequent saccharification step when compared to biomass that was not pH adjusted prior to pretreatment (control biomass). In some embodiments, the biomass comprises corn stover.

As described in the examples, it has been discovered that the use of Mg(OH)₂ to adjust the pH of the biomass before pretreatment decreases the amount of saccharification inhibitors during the saccharification step. For example, in some embodiments, the amount of the inhibitor 5-HMF (5-(Hydroxymethyl)furfural) produced during the saccharification step is reduced in biomass that is pH-adjusted with Mg(OH)₂, e.g., by at least about 10, 20, 30, 40, or 50 mg/ml or more, as compared to the amount of 5-HMF produced during the saccharification step in biomass that is not pH-adjusted with Mg(OH)₂. In other embodiments, the amount of the inhibitor furfural produced during the saccharification step is reduced in biomass that is pH-adjusted with Mg(OH)₂, e.g., by at least about 10, 20, 50, 100, 200, or 300 mg/L or more, as compared to the amount of furfural produced during the saccharification step in biomass that is not pH-adjusted with Mg(OH)₂.

In some embodiments, the amount of ferulic acid produced during the saccharification step is reduced in biomass pH-adjusted with Mg(OH)₂, e.g., by at least about 5, 10, 15, 20, 25, 30, 35, 40, 45, 50, 60, 70 mg/L or more, compared to the amount of ferulic acid produced during the saccharification step in biomass that is not pH-adjusted with Mg(OH)₂. The reduction in the amount of ferulic acid suggests that ferulic acid esterase (FAE) may be less active in the Mg(OH)₂ treated biomass. Increasing the amount of FAE, for example, by use of a yeast strain expressing FAE, may increase the amount of saccharification.

### pH Adjustment After HPHT

In another aspect, the pH of the biomass can be adjusted after the HPHT pretreatment step to be within the optimal range for activity of saccharification enzymes, e.g., within the range of about 4.0 to 6.0. Thus, in some embodiments, the unadjusted pH (i.e., prior to addition of Mg(OH)₂) of biomass after the HPHT step is in the range of about 3.8 to 4.5. Therefore, in some embodiments, the pH is adjusted (e.g., increased at least 0.2, 0.4, or more pH units) to about 5.0, or at least about 5.0, with Mg(OH)₂. In other embodiments, the pH is adjusted (e.g., increased at least 0.2, 0.4, or more pH units) to a range of about 4.0 to 6.0, for example, at least about 4.6, 4.7, 4.8, 4.9, 5.0, 5.1, 5.2, 5.3, 5.4 or 5.5 with Mg(OH)₂. In some embodiments, the pH of the HPHT-treated biomass is adjusted (e.g., increased at least 0.2, 0.4, or more pH units) to about 5.0, or at least about 5.0, using both Mg(OH)₂ and NH₄OH. In other embodiments, the pH of the HPHT-treated biomass is adjusted (e.g., increased at least 0.2, 0.4, or more pH units) to a range of about 4.0 to 6.0, for example, at least about 4.6, 4.7, 4.8, 4.9, 5.0, 5.1, 5.2, 5.3, 5.4 or 5.5 using both Mg(OH)₂ and NH₄OH. The combination of Mg(OH)₂ and NH₄OH after HPHT pretreatment may provide for a more stable pH environment during the subsequent simultaneous saccharification and fermentation (SSF) stages when compared to using NH₄OH alone. Further, the combination of Mg(OH)₂ and NH₄OH is expected to improve the function of the yeast in the SSF process by providing magnesium ions and the ammonium nitrogen.

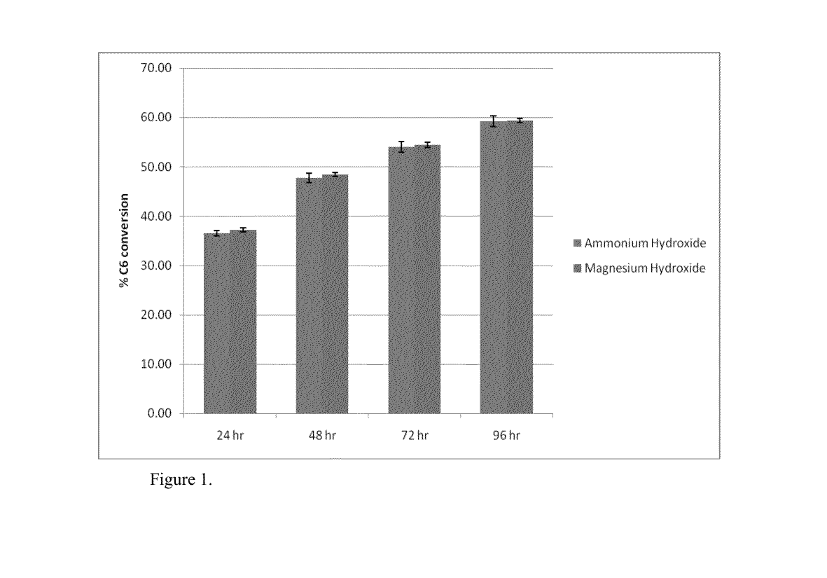

As further described in the Examples, it has been discovered that using Mg(OH)₂ to adjust the pH of the biomass does not result in a significant decrease in the amount of cellulose converted to fermentable sugars when compared to using NH₄OH to adjust the pH. For example, as shown in FIG. 1, when the pH was adjusted using either Mg(OH)₂ or NH₄OH after HPHT pretreatment, similar percent conversion of cellulose to C6 sugars occurred at all time points tested. Thus, Mg(OH)₂ can be used instead of NH₄OH without compromising recovery of fermentable sugars.

### Additional Aspects of the Invention

The methods described herein are useful for producing a pH-adjusted biomass. Examples of types of biomass useful in the methods of the invention include, but are not limited to, agricultural crops, forest crops, and different types of waste and byproducts that contain lignocellulose and/or cellulose. Biomass includes, but is not limited to, agricultural biomass such as corn stover, corn cobs, corn husks, wheat straw, rice straw, rice hulls, barley straw, oat straw, oat hulls, canola straw, and soybean stover; grasses such as switchgrass, miscanthus, cord grass, rye grass, and reed canary grass; sugar cane and sugar processing byproducts, such as baggase and beet pulp; wood products, trees and parts thereof, sawdust, recycled pulp fiber, wood chips, newsprint, and cardboard; and animal waste. The biomass may also comprise a processed lignocellulosic feedstock.

Examples of enzymes that are useful in saccharification of lignocellulosic biomass include glycosidases, cellulases, hemicellulases, starch-hydrolyzing glycosidases, xylanases, ligninases, and feruloyl esterases, and combinations thereof. Glycosidases hydrolyze the ether linkages of di-, oligo-, and polysaccharides. The term cellulase is a generic term for a group of glycosidase enzymes which hydrolyze cellulose to glucose, cellobiose, and other cello-oligosaccharides. Cellulase can include a mixture comprising exo-cellobiohydrolases (CBH), endoglucanases (EG) and β-glucosidases (βG). Specific examples of saccharification enzymes include carboxymethyl cellulase, xylanase, β-glucosidase, β-xylosidase, and α-L-arabinofuranosidase, and amylases. Saccharification enzymes are commercially available, for example, Fiberzymes™ (EdeniQ, Visalia, Calif.), Cellic® CTec2 and HTec2 (Novozymes, Denmark), Spezyme® CP cellulose (Genencor International, Rochester, N.Y.) and Multifect® xylanase (Genencor). Saccharification enzymes can also be expressed by host organisms, including recombinant microorganisms.

The saccharification reaction can be performed at or near the temperature and pH optimum for the saccharification enzymes used. In some embodiments of the present methods, the temperature optimum for saccharification ranges from about 15 to about 100° C. In other embodiments, the temperature range is about 20 to 80° C., about 35 to 65° C., about 40 to 60° C., about 45 to 55° C., or about 45 to 50° C. The pH optimum for the saccharification enzymes can range from about 2.0 to 11.0, about 4.0 to 6.0, about 4.0 to 5.5, about 4.5 to 5.5, or about 5.0 to 5.5, depending on the enzyme.

The enzyme saccharification reaction can be performed for a period of time from about several minutes to about 250 hours, or any amount of time between. For example, the saccharification reaction time can be about 5 minutes, 10 minutes, 30 minutes, 60 minutes, or 2, 4, 6, 8, 12, 16, 18, 24, 36, 48, 60, 72, 84, 96, 108, 120, 130, 140, 150, 160, 170, 180, 190, 200, 210, 220, 230, 240 or 250 hours. In other embodiments, the saccharification reaction is performed with mixing or shaking to improve access of the enzymes to the cellulose. The mixing or shaking can be, for example, at 50 to 250 rpm.

The amount of saccharification enzymes added to the reaction can be adjusted based on the cellulose content of the biomass and/or the amount of solids present in a composition comprising the biomass, and also on the desired rate of cellulose conversion. For example, in some embodiments, the amount of enzymes added is based on % by weight of cellulose present in the biomass, as specified by the enzyme provider(s). The % of enzyme added by weight of cellulose in such embodiments can range from about 0.1% to about 10% on this basis.

In some embodiments, the biomass is combined with water to produce a composition comprising water and solids. The amount of solids in the biomass composition can be about 5% to about 50%, about 10% to about 40%, about 15% to about 35%, or about 18% to about 32%. In some embodiments, the amount of solids in the biomass composition is at least 5, 10, 15, 16, 17, 18, 19, 20, 21, 22, 23, 24, 25, 30, 35, 40, 45, or 50% or more solids. The amount of solids can be adjusted as desired both before and after the pretreatment step. The amount of saccharification enzymes added can be adjusted based on the solid content of the biomass composition.

The methods described herein may be performed as a batch process or continuous process.

One advantage of the present methods is that an acid pretreatment step can be avoided if desired. Acid pretreatment can degrade sugars produced during the pretreatment stage into inhibitory compounds. Acid pretreatment can also lead to the deposition of insoluble salts (“scale”) on equipment downstream in the process. Adjustment of the pH following acid treatment to the pH range of 4.0 to 6.0 has the disadvantage of increasing the deposition of salts on equipment. Thus, in some embodiments, the present method does not make use of an acid pretreatment, and therefore avoids the above described problems. Accordingly, an “acid-free” method involves a method in which no or substantially no exogenous acid is added to the material.

For example, the acid free method of the invention provides biomass that has not been treated with an acid prior to the HPHT pretreatment step, such that the pH of the biomass is in the range of from about 4.0 to 8.0 prior to HPHT pretreatment. For example, in some embodiments, the pH of the biomass prior to the pretreatment step and without acid treatment is about 4.0, 4.5, 5.0, 5.5, 6.0, 6.5, 7.0, 7.5 or 8.0. In some embodiments, the acid free method of the invention also provides biomass that has not been treated with an acid either during and after the HPHT step. For example, in some embodiments, the pH of the biomass is between about 4.0 and 8.0 during the HPHT step, for example, between about 4.0 and 6.0, or between about 4.2 and 5.2. In other embodiments, the pH of the biomass after HPHT pretreatment is between about 4.0 and 6.0 for biomass that was pH adjusted with Mg(OH)₂ prior to the pretreatment step. In certain embodiments, the pH of the acid-free biomass that was not pH adjusted with Mg(OH)₂ is in the range of about 4.0 to 6.0 before pretreatment and in the range of about 3.5 to about 6.0 after HPHT pretreatment. For example, in some embodiments, the pH after pretreatment and prior to pH adjustment with Mg(OH)₂ is between about 3.5 and 6.0, for example, between about 3.6 and 5.5, or between about 3.8 and 5.0, or any range in between. Further, in some embodiments, the acid-free biomass is not subjected to flashing steps after HPHT pretreatment and prior to the saccharification step.

The present methods also do not require pretreatment of the lignocellulosic biomass with metal carbonates prior to treatment with Mg(OH)₂. Such methods are described in U.S. patent application Ser. No. 12/621,599 (US 2010/0124771) to Sabesan et al.

### EXAMPLES

### Example 1

### Comparison of Ammonium Hydroxide and Magnesium Hydroxide for Adjusting the pH of the Biomass

This example compares the use of ammonium hydroxide and magnesium hydroxide for adjusting the pH of the biomass comprising corn stover after HPHT pretreatment.

Traditionally, NH₄OH was used to adjust the pH of corn stover, switchgrass and wood chips after HPHT. The amount of NH₄OH ranges from ˜900 mls with 20% corn stover solids to ˜1100 mls with 20% wood chips (citrus wood) although at times larger amounts have been necessary.

The goal of the present experiment was to determine the amount of Mg(OH)₂ sufficient to effectively gain the same amount of pH adjustment as NH₄OH.

Pilot Plant pH adjustment using NH₄OH:

900 ml of NH₄OH was added to approximately 495 lbs of biomass at ˜19.7% solids at a starting pH of 4.28. Density of NH₄OH is 0.895 g/cc (30% NH3). The pH of the biomass after addition of NH₄OH was 5.10.

The amount of NH₄OH used per gram of corn stover mixture was calculated as follows: 900 ml NH₄OH×0.895 g/cc/495 lbs biomass×454 g/lb=0.003584 g×1000 mg/g=3.584 mg/gram corn stover.

Laboratory pH adjustment using Mg(OH)₂:

Mg(OH)₂ has two hydroxides when it dissociates, therefore, theoretically if, for example, one needs 3.584 mg of NH₄OH per gram of corn stover, one should be able to use half as many grams of Mg(OH)₂ to obtain the same pH change as NH₄OH.

The lab scale experiment used 150 g of corn stover that was pretreated with HPHT. The pH after pretreatment was 4.18. The pH was adjusted using 2 mg of Mg(OH)₂ per gram of corn stover. Mixing was facilitated with a propeller drill bit, and the pH of the biomass after addition of Mg(OH)₂ was 5.00.

In summary, the same pH change was observed in the pilot plant using NH₄OH to adjust the pH and in the lab using Mg(OH)₂ to adjust the pH. This example shows that the use of Mg(OH)₂ provides an effective means of pH adjustment while reducing the risk/safety hazard of using high volumes of NH₄OH. Using Mg(OH)₂ to adjust the pH has the added benefit of adding magnesium, which helps in the saccharification process, without having to add exogenous magnesium, such as magnesium sulfate. Small volumes of NH₄OH can be added if required by the yeast during fermentation.

### Example 2

### Magnesium Hydroxide Use in Citrus Wood Saccharification

This example describes the use of magnesium hydroxide to adjust the pH of biomass comprising citrus wood after pretreatment.

This test compared laboratory saccharification of citrus wood when the material was pH adjusted with magnesium hydroxide to pH 5.0 following HPHT/Cellunator™ pretreatment. Test samples consisted of ˜20% citrus wood solids. The standard HPHT/Cellunator™ pretreatment was performed as described in Examples 4-7.

Following HPHT/Cellunator™ pretreatment, the material was adjusted to approximately 16.2% solids (to facilitate agitation in the shaking incubator). The starting pH of the material prior to adjustment by either ammonium hydroxide or magnesium hydroxide was approximately 4.07. The pretreated material was adjusted to pH 5.0 with either ammonium hydroxide (3 ml of 28% solution) or magnesium hydroxide (1.5 g/kg). Standard saccharification at 104 degrees F. (40° C.), 150 rpm and using Ctec II/Htec II enzymes (10% Ctec II by cellulose content and 0.5% Htec II by solids) was performed on the pH adjusted pretreated material.

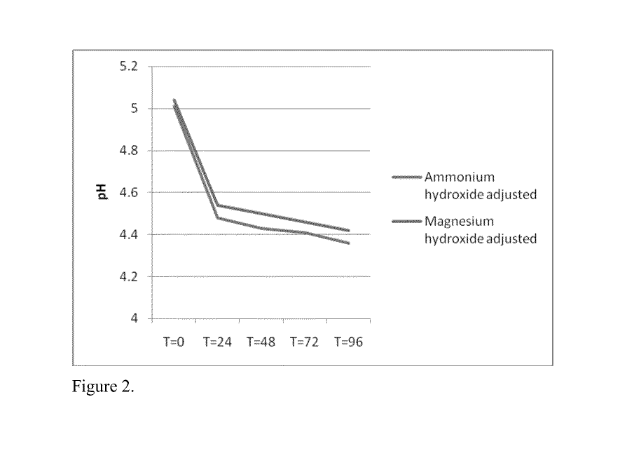

Results: As shown in FIG. 1, the conversion of C6 sugars was substantially the same in the biomass that was pH adjusted with either ammonium hydroxide or magnesium hydroxide after HPHT pretreatment. Further, as shown in Table 1, the production of inhibitors during the saccharification process was substantially similar between the two treatments. Moreover, as shown in FIG. 2, the pH change during saccharification was similar between the two treatments.

TABLE 1
Production of Inhibitors in Biomass that was pH adjusted with NH₄OH or Mg(OH)_{2.}*

| | NH₄OH | NH₄OH | NH₄OH | NH₄OH | Mg(OH)₂ | Mg(OH)₂ | Mg(OH)₂ | Mg(OH)₂ |
|---|---|---|---|---|---|---|---|---|
| Sample | T = 0 | T = 24 | T = 48 | T = 64.5 | T = 0 | T = 24 | T = 48 | T = 86 |
| Analysis | mg/L | | | | | | | |
| 2-Furoic acid | 94.4 | 99.5 | 100.1 | 98.8 | 91.2 | 83.1 | 84 | 80.7 |
| 5-HMF | 218.5 | 176.3 | 173.5 | 170.7 | 244.2 | 216.3 | 212 | 202.8 |
| Furfural | 716.7 | 657.2 | 630.3 | 627.4 | 571.4 | 490.5 | 521.1 | 501.6 |
| 4-HBA | 15.4 | 24.7 | 0.5 | 0.4 | 15.3 | 0.8 | 0.6 | 0.3 |
| Syringic acid | 23.6 | 17.7 | 18 | 16.7 | 22.7 | 22.8 | 22.7 | 18.6 |
| Vanillin | 9.9 | 8 | 7.8 | 8 | 9.6 | 11.6 | 12.3 | 8 |
| Syringaldehyde | 1.1 | 0.9 | 0.9 | 0.9 | 1.1 | 3.2 | 1.9 | 1.7 |
| p-Coumaric acid | 17.6 | 17.2 | 16.2 | 17 | 16.8 | 21.6 | 22.8 | 15.4 |
| Ferulic acid | 2.7 | 1.8 | 0.8 | 2.8 | 2.4 | 12.4 | 15.8 | 5.5 |

*Citrus wood, post-HPHT pH adjustment; 20% solids at 148 psig and 5 minutes residence time. Starting pH: NH₄OH = 4.13; Mg(OH)₂ = 4.07.

This example demonstrates that magnesium hydroxide can be substituted for ammonium hydroxide to adjust the pH to 5.0 after the HPHT pretreatment step without significant alterations to the saccharification parameters. This allows for the use of a safer chemical, namely magnesium hydroxide, for pH adjustment.

### Example 3

### Magnesium Hydroxide Use in Corn Stover Pretreatment

This example describes the use of magnesium hydroxide to adjust the pH of biomass comprising corn stover prior to pretreatment.

This test compared corn stover treated at a high temperature and pressure with and without the addition of magnesium hydroxide. The goal was to determine if pH adjustment before HPHT alters inhibitor production and saccharification yields.

Test samples consisted of 20% corn stover solids (400 g water and 100 g corn stover). The small laboratory HPHT reactor was used at the following conditions: Temp. 343 degrees F. (173° C.) and pressure at 125 psi.

The first run was a standard HPHT at the above described conditions (without use of magnesium hydroxide). The pre-HPHT pH was ˜6.2 and the ending pH was 4.26.

The second run tested the addition of magnesium hydroxide prior to the start of the HPHT run.

The pre-HPHT pH was ˜6.17. Following the addition of magnesium hydroxide (880 mg; 2.2 mg Mg(OH)₂/gram of corn stover solids), the pH of the material was 7.54. After HPHT, the ending pH was 4.61.

Standard saccharification at 104 degrees F. (40° C.), 150 rpm and using Ctec II/Htec II enzymes was performed on the pretreated material.

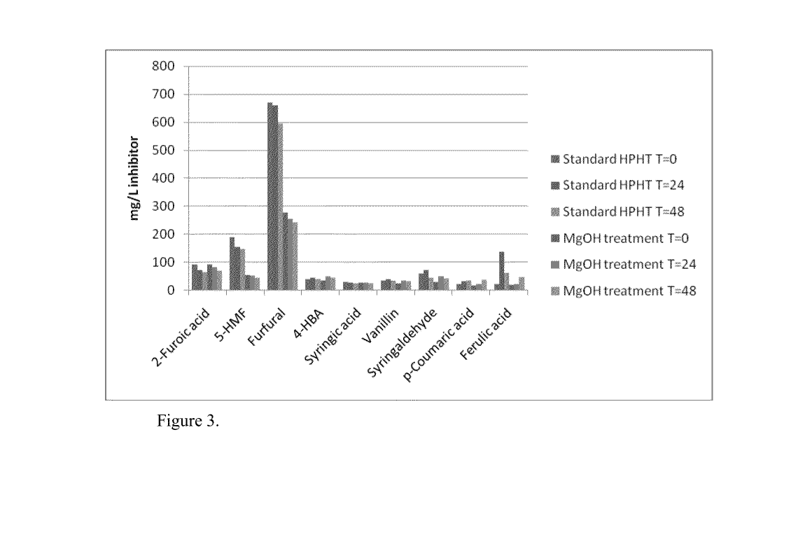

Results: Corn stover pH-adjusted with magnesium hydroxide prior to HPHT pretreatment had increased conversion of biomass to C6 sugars at the 24 hour time-point compared to biomass that was not pH adjusted prior to HPHT pretreatment. The percent conversion to C6 sugars was slightly less than the non-pH adjusted material at the 48 and 72 hour time-points. The conversion of biomass to C5 sugars was similar between the two treatments at the 24 hour time-point, and slightly less than the non-pH adjusted material at the 48 and 72 hour time-points. As shown in FIG. 3, the amount of inhibitors 5-HMF and furfural was substantially lower in the magnesium hydroxide pH-adjusted material. Interestingly, at T=24 hours, the ferulic acid levels in the magnesium hydroxide treated material are significantly lower. This suggests that ferulic acid esterase may be less active in the magnesium hydroxide treated material.

This example demonstrates that pretreatment of corn stover solids with magnesium hydroxide prior to HPHT results in lower levels of the inhibitors furfural and 5-HMF. Saccharification of magnesium hydroxide pretreated corn stover is increased at T=24 hours relative to the biomass not treated with magnesium hydroxide.

### Example 4

The following examples describe variations of the process that can be used to adjust the pH of the biomass using Mg(OH)₂.

Process description #1—Introduction of Mg(OH)₂ before pre-treatment—use of Cellunator™ after HPHT (HPHT=High Pressure High Temperature)
Pre wet Biomass with water.
Fill the pre-treatment tank with the appropriate amount of water as needed for the process.
Start agitation and circulation.
Bring water temperature to 170 to 190 deg F. and maintain that temp range.
Add calculated amount of Mg(OH)₂
   pH starting range is from 7.0 to 7.5 with the addition of Mg(OH)₂
   pH starting range is from 5.5 to 6.2 without the addition of Mg(OH)₂
Start adding pre-wetted Biomass. Adjust pump and agitator speed as necessary to maintain flow at 4 to 10 gallons per minute.
Once all material is added, allow contents to mix/circulate for 20 minutes at 195 deg F.
Bring temperature to 200 deg F., then close the system and start increasing temperature.
Increase temperature and pressure until tank reaches 125 to 145 psi.
Record T and P, hold the tank at this temperature and pressure for 10 minutes.
After 10 to 30 minutes at temp/pressure, start cooling.
Cool slurry to 200 deg F.
If pressure is below 20 psi, relieve pressure by opening valve on the feed port.
Begin post HPHT Cellunator™ run maintaining temperatures at 180-190° F.
   Treat the material using the Cellunator™ for 10 to 30 minutes. As described herein, the Cellunator™ is a colloidal mill prescribed by EdeniQ (Visalia, Calif.) for custom manufacture.
Bring slurry to 110 to 105 deg F.
   pH ending range is from 4.2 to 5.2 with the addition of Mg(OH)₂.
   pH ending range is from 3.8 to 4.5 without the addition of Mg(OH)₂.

### Example 5

Process description #2—Introduction of Mg(OH)₂ before pre-treatment—use of Cellunator™ during HPHT
Pre wet Biomass with water.
Fill the pre-treatment tank with the appropriate amount of water as needed for the process.
Start agitation and circulation.
Bring water temperature to 170 to 190 deg F. and maintain that temp range.
Add calculated amount of Mg(OH)₂
   pH starting range is from 7.0 to 7.5 with the addition of Mg(OH)₂
   pH starting range is from 5.5 to 6.2 without the addition of Mg(OH)₂
Start adding pre-wetted Biomass. Adjust pump and agitator speed as necessary to maintain flow at 4 to 10 gallons per minute.
Begin Cellunator™ run during solids loading and throughout the process.
Once all material is added, allow contents to mix/circulate for 10 to 30 minutes at 195 deg F.
Bring temperature to 200 deg F., then close the system and start increasing temperature.
Increase temperature and pressure until tank reaches 125 to 145 psi.
Record T and P, hold the tank at this temperature and pressure during Cellunator™ run at the desired set-points.
   Treat the material at high temperature and pressure using the Cellunator™ for 10 to 30 minutes
After the Cellunator™ process, shut down Cellunator™ and start cooling.
Cool slurry to 200 deg F.
If pressure is below 20 psi, relieve pressure by opening valve on the feed port.
Bring slurry to 110 to 105 deg F.
   pH ending range is from 4.2 to 5.2 with the addition of Mg(OH)₂
   pH ending range is from 3.8 to 4.5 without the addition of Mg(OH)₂

### Example 6

Process description #3—Introduction of Mg(OH)₂ after pre-treatment
Pre wet Biomass with water.
Fill the pre-treatment tank with the appropriate amount of water as needed for the process.
Start agitation and circulation.
Bring water temperature to 170 to 190 deg F. and maintain that temp range.
Start adding pre-wetted Biomass. Adjust pump and agitator speed as necessary to maintain flow at 4 to 10 gallons per minute.
Begin Cellunator™ run during solids loading and throughout the process.
Once all material is added, allow contents to mix/circulate for 10 to 30 minutes at 195 deg F.
Bring temperature to 200 deg F., then close the system and start increasing temperature.
Increase temperature and pressure until tank reaches 125 to 145 psi.
Record T and P, hold the tank at this temperature and pressure during Cellunator™ run at the desired set-points.
   Treat the material at high temperature and pressure using the Cellunator™ for 10 to 30 minutes
After Cellunator™ process, shut down Cellunator™ and start cooling.
Cool slurry to 200 deg F.
If pressure is below 20 psi, relieve pressure by opening valve on the feed port.
Bring slurry to 110 to 105 deg F.
   pH starting range is from 3.8 to 4.5 after HPHT/Cellunation
   pH is adjusted to 5.0 with the addition of Mg(OH)₂.

### Example 7

Process description #4—Introduction of Mg(OH)₂ and NH₄OH after pre-treatment
Pre wet Biomass with water.
Fill the pre-treatment tank with the appropriate amount of water as needed for the process.
Start agitation and circulation.
Bring water temperature to 170 to 190 deg F. and maintain that temp range.
Start adding pre-wetted Biomass. Adjust pump and agitator speed as necessary to maintain flow at 4 to 10 gallons per minute.
Begin Cellunator™ run during solids loading and throughout the process.
Once all material is added, allow contents to mix/circulate for 10 to 30 minutes at 195 deg F.
Bring temperature to 200 deg F., then close the system and start increasing temperature.
Increase temperature and pressure until tank reaches 125 to 145 psi.
Record T and P, hold the tank at this temperature and pressure during Cellunator™ run at the desired set-points.
   Treat the material at high temperature and pressure using the Cellunator™ for 10 to 30 minutes
After Cellunator™ process, shut down Cellunator™ and start cooling.
Cool slurry to 200 deg F.
If pressure is below 20 psi, relieve pressure by opening valve on the feed port.
Bring slurry to 110 to 105 deg F.
   pH starting range is from 3.8 to 4.5 after HPHT/Cellunation
   pH is adjusted to 5.0 with the addition of Mg(OH)₂ and NH₄OH

The above examples show that adjustment of pH using Mg(OH)₂ before or after the HPHT/Cellunator™ process allows for a more stable pH environment during saccharification. The buffering capacity of the Mg(OH)₂ maintains the pH of the simultaneous saccharification and fermentation (SSF) between about 4.7 and 5.0 (optimal ranges for the activity of the Cellulosic enzymes).

Adjustment of pH using both Mg(OH)₂ and NH₄OH after the HPHT/Cellunator™ process will allow for a more stable pH environment as opposed to the use of NH₄OH alone. By using NH₄OH in combination with Mg(OH)₂, the yeast being used in the SSF process will benefit both from the magnesium ions being present and the ammonium nitrogen

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for processing biomass containing a cellulosic material, comprising:
a) contacting biomass below pH 6.5 with a sufficient amount of magnesium hydroxide (Mg(OH)₂) to increase the pH of the biomass above pH 7.0;
b) treating the biomass comprising Mg(OH)₂ at an elevated temperature and pressure;
c) contacting the treated biomass with saccharification enzymes under conditions sufficient to hydrolyze at least a portion of the cellulose to fermentable sugars.

2. The method of claim 1, wherein the pH of the biomass in step (a) is increased from a range of about pH 5.5 to 6.2.

3. The method of claim 1, wherein the pH of the biomass in step (a) is increased to a range of about pH 7.0 to 8.0.

4. The method of claim 1, further comprising, following step b), contacting the treated biomass with a sufficient amount of Mg(OH)₂ to adjust the pH to about 4.0-6.0.

5. The method of claim 1, wherein the treating comprises treating the biomass comprising Mg(OH)₂ at a temperature greater than about 160° F.

6. The method of claim 5, wherein the treating comprises treating the biomass comprising Mg(OH)₂ at a pressure of greater than about 120 psi.

7. The method of claim 1, wherein the biomass contains less than 1% by weight of a metal carbonate.

8. The method of claim 1, wherein step (c) comprises contacting the biomass with yeast cells that express the saccharification enzymes.

9. The method of claim 1, further comprising mechanically reducing biomass particle size either before, during, or after the treating.

10. The method of claim 9, wherein at least 85% of the biomass particles by weight have a particle size from about 100 microns to about 800 microns.

11. An acid-free method for processing biomass containing a cellulosic material, comprising:
a) contacting the biomass with magnesium hydroxide (Mg(OH)₂) at a concentration sufficient to raise the pH of the biomass at least 0.2 pH units to between pH 4.0 and 8.0;
b) treating the biomass comprising Mg(OH)₂ at an elevated temperature and pressure; and
c) contacting the treated biomass following a) and b) with saccharification enzymes under conditions sufficient to convert at least a portion of the cellulose to fermentable sugars.

12. The method of claim 11, wherein step a) occurs before step b).

13. The method of claim 12, wherein step a) increases the pH of the biomass at least 0.5 pH units to between pH 7.0-8.0.

14. The method of claim 11, wherein step b) occurs before step a).

15. The method of claim 14, wherein step a) increases the pH of the biomass at least 0.5 pH units to between pH 3.5-5.0.

16. The method of claim 11, wherein the contacting further comprises contacting the biomass with ammonium hydroxide (NH₄OH).

17. The method of claim 11, wherein the treating comprises treating the biomass comprising Mg(OH)₂ at a temperature greater than about 160° F.

18. The method of claim 17, wherein the treating comprises treating the biomass comprising Mg(OH)₂ at a pressure of greater than about 120 psi.

19. The method of claim 11, further comprising mechanically reducing biomass particle size either before, during, or after the treating.

20. The method of claim 19, wherein at least 85% of the biomass particles by weight have a particle size from about 100 microns to about 800 microns.
